(19) United States
(12) Patent Application Publication
(10) Pub. No.: US 2023/0389972 A1
(43) Pub. Date: Dec. 7, 2023

(54) BONE SCREW EXTRACTION DEVICE AND METHODS OF USE THEREOF

(71) Applicant: Arizona Board of Regents on Behalf of the University of Arizina, Tucson, AZ (US)

(72) Inventors: L. Daniel Latt, Tucson, AZ (US); Carlos Urrea-De La Puerta, Tucson, AZ (US); Carolina Gomez Llanos, Tucson, AZ (US); Emilio Araiza, Tucson, AZ (US); Erick De Leon, Tucson, AZ (US); Eva Richter, Tucson, AZ (US)

(21) Appl. No.: 18/310,526

(22) Filed: May 1, 2023

Related U.S. Application Data
(60) Provisional application No. 63/337,142, filed on May 1, 2022.

(51) Int. Cl. A61B 17/88

(52) U.S. Cl. CPC A61B 17/8877

(57) ABSTRACT

A surgical instrument for the nondestructive removal of screws or fasteners, the device includes an elongated body having a first end including a handle portion and a second end including a screw engaging portion, the first end is offset at an angle to the second end, the screw engaging portion is a bifurcated protrusion that includes a first part and a second part wherein the first and second parts define a generally V-shaped groove; the protrusion has an exterior surface that is biocompatible and retains a cutting edge, the instrument is configured for impaction and bending resistance.

### CROSS REFERENCE TO RELATED APPLICATIONS

This application claims the benefit of the filing date of U.S. Provisional Application No. 63/337,142, filed on May 1, 2022, entitled “BONE SCREW EXTRACTION DEVICE AND METHODS OF USE THEREOF.” The entirety of the foregoing is hereby incorporated by reference.

### FIELD OF THE INVENTION

The field of this invention generally relates to an improved method and apparatus for the nondestructive removal of surgical screws from mammalian subjects.

### BACKGROUND OF THE INVENTION

In 2017, approximately 22.3 million orthopedic surgeries were performed throughout the world. Certain common orthopedic surgeries (e.g., hip repair surgery, spinal vertebral fusion surgery) involve the use of screws to stabilize bones and/or bone fragments during the healing process. Post-operative complications, such as hardware failure and infection can occur at the surgical site. Follow-up surgery is often needed to remove loose hardware (e.g., bone screws) and infected tissue.

Devices, kits and methods of using same devices and kits are available to extract screws from bones at surgical sites. However, surgeons continue to encounter problems when attempting to extract screws from biological tissue (e.g., bone). Accordingly, there is still a need for improved devices and methods for extracting surgical screws.

### BRIEF DESCRIPTION OF THE DRAWINGS

FIG. 1 is a perspective view of one embodiment of a device according to the present disclosure.

FIG. 2 is a perspective schematic view of an embodiment of a device of the present disclosure showing longitudinal axis of the handle portion and the screw engaging portion.

FIG. 2a is a side view of the device of FIG. 2.

FIG. 3 is a detail view of the screw engaging portion of the device of FIG. 1.

FIG. 4 is a perspective view of an operator moving the handle portion of the device of FIG in a first direction causing a movement of the screw engaging portion in an opposite direction.

### DESCRIPTION

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

As used herein, the term “biocompatible surface” refers to a surface of an object, the surface having the ability to be in contact with a patient without producing a substantial adverse effect on a tissue or biological system of the patient.

As used herein, the term “patient” refers to a mammalian organism (e.g., a human, a wild or domestic animal) being treated for a medical condition (e.g., a broken bone).

As used herein, the term “handle” refers to a part of an object that may be held, seized, or grasped.

As used herein, the term “cutting surface” refers to a surface whose profile is a decreasing angle configured for incising material without dulling.

As used herein, the term “impact resistant” refers to a material that does not exhibit stress, deformation, bending, cracking, or weakening when struck by another object.

As used herein, the term “manual force” refers to the effort exerted by the user of the apparatus to impart a force by way of mechanical advantage onto a fastener.

The present disclosure generally relates to improved methods and devices for the nondestructive removal of surgical screws from mammalian subjects. “Nondestructive removal”, as used herein, refers to the removal of a surgical screw without substantially degrading (e.g., fragmenting) the surgical screw. The methods and apparatuses are particularly suitable for the removal of solid (e.g., non-cannulated) surgical screws, but may be useful also for the removal of cannulated screws. In addition, the devices and methods of the present disclosure are particularly useful for the removal of screws that are disposed in a bone but are no longer threadably engaged (e.g., have “lost their purchase”) with the bone. “Threadably engaged”, as used herein, refers to screws that are surrounded by tissue (e.g., bone tissue) that has enough integrity such that turning the threaded screw clockwise or counterclockwise relative to its longitudinal axis will result in movement of the screw along its longitudinal axis.

Advantageously, the devices of the present disclosure include features that permit the operator to dislodge and optionally remove tissue surrounding at least a part (e.g., the head) of a surgical screw, thus providing better access to engage the screw with other features of the devices.

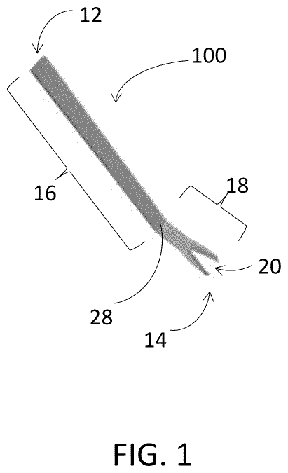

In one aspect, the present disclosure provides a device for extracting a screw that is partially or completely disposed in a tissue (e.g., bone). FIG. 1 show a perspective view of one embodiment of a device 100 according to the present disclosure. The device 100 comprises an elongated body 10 having a first end 12 and a second end 14. The first end 12 comprises a handle portion 16 and the second end comprises a screw engaging portion 18.

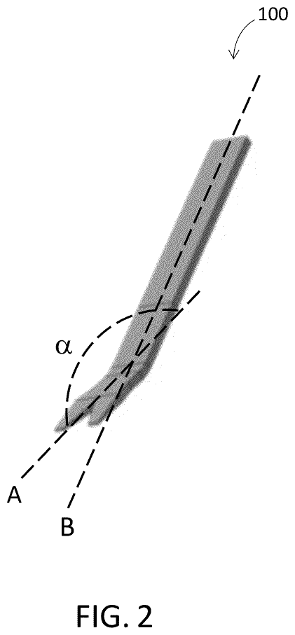

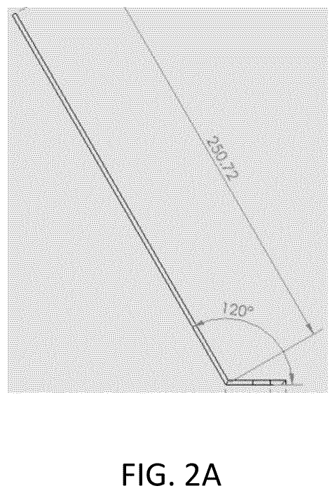

The handle portion 16 of the first end 12 is disposed at an angle (e.g., angle “a”) to the screw engaging portion 18 of the second end 14, as shown in FIG. 2. Accordingly, the handle portion 16 has a first longitudinal axis “A” and the screw engaging portion 18 has a second longitudinal axis “B” that is not coincident with the first longitudinal axis.

In some embodiments, the first end 12 is disposed at an angle to the second end 14 due to a bend 28 or a curvature (not shown) in the device 100. In some embodiments, the first longitudinal axis and the second longitudinal axis intersect to form an angle (“a”) that is greater than or equal to 90°. In some embodiments the first longitudinal axis and the second longitudinal axis intersect to form an angle (“a”) that is about 90-150°.

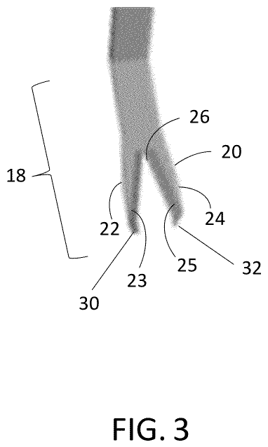

The screw engaging portion 18 comprises a bifurcated protrusion 20. The screw engaging portion 18 is shown in greater detail in FIG. 3. The bifurcated protrusion 20 comprises a first part 22 and a second part 24 which, together, define a generally V-shaped groove 26.

In any embodiment, the device 100 can be fabricated (e.g., by 3-D printing) using a biocompatible material. Nonlimiting examples of biocompatible materials include 304 stainless steel, 316 stainless steel, SAE 316 stainless steel, titanium or titanium alloys, Inconel, low carbon steel, alloys of steel including chromium, nickel, molybdenum, aluminum, austenitic stainless steel, fiber reinforced polymer, and a combination of any two or more of the foregoing materials. Alternatively, or additionally, the material used to fabricate the device can be coated (e.g., during or after fabrication) with a biocompatible coating material.

In any embodiment of the device of the present disclosure, the first part 22 has a first edge 23 that faces the second part 24 and the second part 24 has a second edge 25 that faces the first part 22. The first edge 23 and/or second edge 25 can comprise a bevel 28 that forms a cutting surface. In some embodiments, both the first edge 23 and the second edge 25 comprise a bevel that forms a cutting surface. The cutting surface can be used to dislodge and, optionally, move tissue (e.g., soft tissue, bone, infected tissue) away from the bone screw so that the device can better engage a portion of the screw (e.g., the screw head).

FIG. 2a shows a device 100 wherein the screw engaging portion 18 forms a linear plane. It can be seen in this illustrated embodiment that the first part 22 and second part 24 of the protrusion are coextensive of the linear plane. In some embodiments either the first part 22 or the second part 24 is not coextensive of the plane formed by the screw engaging portion 18 (not shown). In these embodiments, the first part or second part is canted away from the linear plane. Advantageously, this configuration may be used to make the cutting edges more open and accessible to the tissue surrounding a bone screw.

In any embodiment, either the first part 22 and/or the second part 24 taper to spaced-apart points (points 30 and 32, respectively) at the second end 14 of the device 100, as shown in FIG. 2. A distance between the spaced-apart points is about 3 millimeters to about 13 millimeters.

In any embodiment of the device, the handle portion can have a first length and the screw engaging portion can have a second length, wherein the first length is greater than the second length. Advantageously, as the first length to the second length ratio increases, the user gains additional leverage to remove a bone screw.

In any embodiment, a device of the present disclosure further comprises an impact-receiving element (not shown) disposed on the handle portion (e.g., the distal end of the handle). In some embodiments, the impact-receiving element is an integral part of the handle. In alternative embodiments, the impact-receiving element is connected to the handle (e.g., through a slot or a peg). Nonlimiting examples of suitable impact-receiving elements are caps that can be attached (e.g., permanently or detachably attached) to the handle and rods that can be an integral part of the handle (not shown) or can be attached (e.g., permanently or detachably attached) to the handle.

The impact-receiving element is configured to receive forcible contact from an impact apparatus such as a hammer or a mallet, for example. Advantageously, applying force to the impact-receiving element during use, can facilitate engagement of the bone screw into the V-shaped groove and/or dislodgement and/or removal of tissue (e.g., infected tissue) proximate the screw head. Dislodgement and/or removal of the tissue can facilitate removal of the screw using the device of the present disclosure. The impact-receiving element can be made of any suitable material that can translate at least a part of the impact force to the elongated body and b) withstand the impact force without substantially disintegrating or releasing fragments into the surgical area. Suitable materials for the impact-receiving element include but are not limited to a metal or a polymeric material.

Devices of the present disclosure can be produced using processes and materials known in the art. The material used to make the device may influence the process selected to manufacture the device. Non-limiting examples of suitable manufacturing processes include metal stamping, molding, milling, grinding, drilling, and 3-D printing.

In use, devices of the present disclosure can be used to remove solid screws from bones even if the head of the screw is burrowed into the bone. The points and/or cutting edges of the screw engaging portion can be used to cut and remove bone tissue around the screw to provide access so the V-shaped groove can engage the shank or the thread of the screw and pry it out of the bone. A bend or curvature disposed between the handle portion and the screw engaging portion can be used like a fulcrum to lever the screw out of the bone.

In another aspect, the present disclosure provides a method for extracting a solid screw from a bone, the method comprising extracting the solid screw from the bone using the device of any one of the embodiments described herein.

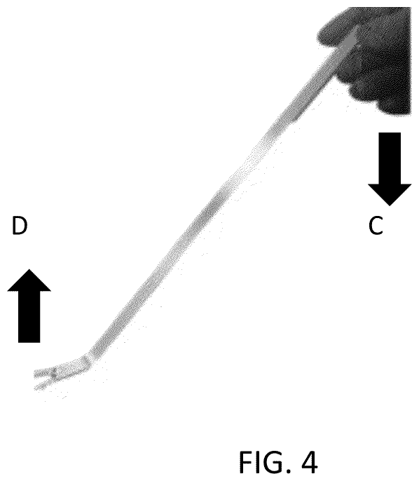

In one implementation, the method comprises engaging a solid bone screw in the V-shaped groove of the device of any one of the device embodiments described herein. Engaging the bone screw comprises moving the screw engaging portion of the device relative to the screw until the screw is in contact, in the V-shaped groove, with the first part and the second part of the protrusion of the device. The method further comprises applying a manual force to the first end of the elongated body to extract the solid screw from the bone. Applying a manual force to the first end can comprise applying a force in a direction opposite the direction the user intends to extract the screw. FIG. 4 shows the application of downward force (designated by arrow “C” to the first end of the device 100. Because the bend 28 of the device 100 can act as a fulcrum, application of the downward force results in movement of the screw engaging portion 18 of the device 100 in the opposite (upward) direction (shown by arrow “D”), resulting in the extraction of the bone screw.

In any implementation, the method further comprises, prior to the applying the manual force to the first end of the body, using at least one of the first part or the second part to dislodge tissue proximate the solid screw.

In any implementation, wherein the device comprises an impact-receiving element on the distal end of the handle portion, the method further comprises contacting an impact apparatus (e.g., a hammer, a mallet) with the impact-receiving element to drive at least a portion of the protrusion into biological tissue proximate the solid screw.

### LIST OF EMBODIMENTS

Embodiment 1. A device, comprising:
   an elongated body having a first end comprising a handle portion and a second end comprising a screw engaging portion;
   wherein the first end is disposed at an angle to the second end;
   wherein the screw engaging portion comprises a bifurcated protrusion that comprises a first part and a second part, the first and second parts defining a generally V-shaped groove;
   wherein the protrusion has an exterior surface that is biocompatible.
Embodiment 2. The device of Embodiment 1, wherein the handle portion has a first longitudinal axis and the screw engaging portion has a second longitudinal axis, wherein the first longitudinal axis is not coincident with the second longitudinal axis.
Embodiment 3. The device of Embodiment 1 or Embodiment 2, wherein the first longitudinal axis and the second longitudinal axis intersect to form an angle that is greater than or equal to 90°.
Embodiment 4. The device of Embodiment 3, wherein the angle is 90-150°.
Embodiment 5. The device of any one of the preceding Embodiments, wherein the first part has a first edge that faces the second part and the second part has a second edge that faces the first part, wherein the first edge or the second edge comprises a bevel that forms a cutting surface.
Embodiment 6. The device of Embodiment 5, wherein both the first edge and the second edge comprise a bevel that forms a cutting surface.
Embodiment 7. The device of any one of the preceding Embodiments, wherein the screw engaging portion defines a plane, wherein neither the first part nor the second part extension is coextensive of the plane.
Embodiment 8. The device of Embodiment 7, wherein the first or the second part is coextensive of the plane.
Embodiment 9. The device of Embodiment 8, wherein the first or the second part is not coextensive of the plane.
Embodiment 10. The device of any one of the preceding Embodiments, wherein either the first part or the second part tapers to a point at the second end.
Embodiment 11. The device of Embodiment 10, wherein the first part and second part taper to spaced-apart points at the second end.
Embodiment 12. The device of Embodiment 11, where a distance between the spaced-apart points is about 3 mm to about 13 mm.
Embodiment 13. The device of any one of the preceding Embodiments, wherein the device is formed of a material selected from the group consisting of 304 stainless steel, 316 stainless steel, SAE 316 stainless steel, titanium or titanium alloys, Inconel, low carbon steel, alloys of steel including chromium, nickel, molybdenum, aluminum, austenitic stainless steel, and a combination of any two or more of the foregoing materials.
Embodiment 14. The device of any one of the preceding Embodiments, wherein the handle portion has a first length, wherein the screw engaging portion has a second length, wherein the first length is greater than the second length
Embodiment 15. A device, comprising:
   an elongated body having a first end comprising a handle portion and a second end comprising a screw engaging portion;
   wherein the first end is disposed at an angle to the second end;
   wherein the screw engaging portion comprises a bifurcated protrusion that comprises a first part and a second part, the first and second parts defining a generally V-shaped groove;
   wherein the screw engaging portion defines a plane, wherein one of the first or the second part is coextensive of the plane, one of the first or the second part is not coextensive of the plane.
Embodiment 16. The device of Embodiment 15, wherein the protrusion has an exterior surface that is biocompatible.
Embodiment 17. The device of any one of the preceding Embodiments, further comprising an impact-receiving element disposed on the distal end of said handle portion configured to receive forcible contact from an impact apparatus.
Embodiment 18. A method for extracting a solid screw from a bone; the method comprising extracting the solid screw from the bone using the device of any one of Embodiments 1 through 14.
Embodiment 19. The method of Embodiment 18, wherein the method comprises:
   engaging the solid screw in the V-shaped groove of the device of any one of Embodiments 1 through 16; and
   applying a manual force to the first end of the elongated body to extract the solid screw from the bone.
Embodiment 20. The method of Embodiment 19, further comprising, prior to the applying the manual force to the first end of the body, using at least one of the first part or the second part to dislodge tissue proximate the solid screw.
Embodiment 21. The method of any one of Embodiments 18 through 20, wherein the device comprises an impact-receiving element on the distal end of the handle portion, wherein the method further comprises contacting an impact apparatus with the impact-receiving element to drive at least a portion of the protrusion into biological tissue proximate the solid screw.

A method, comprising:
engaging a bone screw at least partially disposed in a bone, wherein engaging the bone screw comprises engaging the screw in the V-shaped groove of the device of any one of Embodiments 1-17;
applying a force to the handle portion of the device such that the direction of the force is translated by the device into a direction substantially opposite a direction the screw is oriented into the bone;
while applying the force, engaging a head of the screw with a tools such as a bone screwdriver and rotating the tool in a direction to unscrew the screw from bone.

### LIST OF EMBODIMENTS

In some embodiments the invention encompasses the following non-limiting list of embodiments:
1. A device, comprising:
   an elongated body having a first end comprising a handle portion and a second end comprising a screw engaging portion;
   wherein the first end is disposed at an angle to the second end;
   wherein the screw engaging portion comprises a bifurcated protrusion that comprises a first part and a second part, the first and second parts defining a generally V-shaped groove;
   wherein the protrusion has an exterior surface that is biocompatible.
2. The device of embodiment 1, wherein the handle portion has a first longitudinal axis and the screw engaging portion has a second longitudinal axis, wherein the first longitudinal axis is not coincident with the second longitudinal axis.
3. The device of embodiment 1 or embodiment 2, wherein the first longitudinal axis and the second longitudinal axis intersect to form an angle that is greater than or equal to 90°.
4. The device of embodiment 3, wherein the angle is 90-150°.
5. The device of any one of the preceding embodiments, wherein the first part has a first edge that faces the second part and the second part has a second edge that faces the first part, wherein the first edge or the second edge comprises a bevel that forms a cutting surface.
6. The device of embodiment 5, wherein both the first edge and the second edge comprise a bevel that forms a cutting surface.
7. The device of any one of the preceding embodiments, wherein the screw engaging portion defines a plane, wherein neither the first part nor the second part extension is coextensive of the plane.
8. The device of embodiment 7, wherein the first or the second part is coextensive of the plane.
9. The device of embodiment 8, wherein the first or the second part is not coextensive of the plane.
10. The device of any one of the preceding embodiments, wherein either the first part or the second part tapers to a point at the second end.
11. The device of embodiment 10, wherein the first part and second part taper to spaced-apart points at the second end.
12. The device of embodiment 11, where a distance between the spaced-apart points is about 3 mm to about 13 mm.
13. The device of any one of the preceding embodiments, wherein the device is formed of a material selected from the group consisting of 304 stainless steel, 316 stainless steel, SAE 316 stainless steel, titanium or titanium alloys, Inconel, low carbon steel, alloys of steel including chromium, nickel, molybdenum, aluminum, austenitic stainless steel, and a combination of any two or more of the foregoing materials.
14. The device of any one of the preceding embodiments, wherein the handle portion has a first length, wherein the screw engaging portion has a second length, wherein the first length is greater than the second length.
15. A device, comprising:
   an elongated body having a first end comprising a handle portion and a second end comprising a screw engaging portion;
   wherein the first end is disposed at an angle to the second end;
   wherein the screw engaging portion comprises a bifurcated protrusion that comprises a first part and a second part, the first and second parts defining a generally V-shaped groove;
   wherein the screw engaging portion defines a plane, wherein one of the first or the second part is coextensive of the plane, one of the first or the second part is not coextensive of the plane.
16. The device of embodiment 15, wherein the protrusion has an exterior surface that is biocompatible.
17. The device of any one of the preceding embodiments, further comprising an impact-receiving element disposed on the distal end of said handle portion configured to receive forcible contact from an impact apparatus.
18. A method for extracting a solid screw from a bone; the method comprising extracting the solid screw from the bone using the device of any one of embodiments 1 through 14.
19. The method of embodiment 18, wherein the method comprises:
   engaging the solid screw in the V-shaped groove of the device of any one of embodiments 1 through 16; and
   applying a manual force to the first end of the elongated body to extract the solid screw from the bone.
20. The method of embodiment 19, further comprising, prior to the applying the manual force to the first end of the body, using at least one of the first part or the second part to dislodge tissue proximate the solid screw.
21. The method of any one of embodiments 18 through 20, wherein the device comprises an impact-receiving element on the distal end of the handle portion, wherein the method further comprises contacting an impact apparatus with the impact-receiving element to drive at least a portion of the protrusion into biological tissue proximate the solid screw.
22. A method, comprising:
   engaging a bone screw at least partially disposed in a bone, wherein engaging the bone screw comprises engaging the screw in the V-shaped groove of the device of any one of Embodiments 1-17;
   applying a force to the handle portion of the device such that the direction of the force is translated by the device into a direction substantially opposite a direction the screw is oriented into the bone;
   while applying the force, engaging a head of the screw with a tool such as a bone screwdriver and rotating the tool in a direction to unscrew the screw from bone.

### 3.0 EXAMPLES

The following examples are provided solely to illustrate the present invention and are not intended to limit the scope of the invention, described herein.

Example 1. A Bone Screw Extraction Device similar to the device of FIG. 1 was constructed of 316L stainless steel using a CNC machining process.

All publications mentioned herein are incorporated by reference to the extent they support the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

We claim:

1. A device, comprising:
an elongated body having a first end comprising a handle portion and a second end comprising a screw engaging portion;
wherein the first end is disposed at an angle to the second end;
wherein the screw engaging portion comprises a bifurcated protrusion that comprises a first part and a second part, the first and second parts defining a generally V-shaped groove;
wherein the protrusion has an exterior surface that is biocompatible.

2. The device of claim 1, wherein the handle portion has a first longitudinal axis and the screw engaging portion has a second longitudinal axis, wherein the first longitudinal axis is not coincident with the second longitudinal axis.

3. The device of claim 1, wherein the first longitudinal axis and the second longitudinal axis intersect to form an angle that is greater than or equal to 90°.

4. The device of claim 3, wherein the angle is 90-150°.

5. The device of claim 4, wherein the first part has a first edge that faces the second part and the second part has a second edge that faces the first part, wherein the first edge or the second edge comprises a bevel that forms a cutting surface.

6. The device of claim 5, wherein both the first edge and the second edge comprise a bevel that forms a cutting surface.

7. The device of claim 6, wherein the screw engaging portion defines a plane, wherein neither the first part nor the second part extension is coextensive of the plane.

8. The device of claim 7, wherein the first or the second part is coextensive of the plane.

9. The device of claim 8, wherein the first or the second part is not coextensive of the plane.

10. The device of claim 9, wherein either the first part or the second part tapers to a point at the second end.

11. The device of claim 10, wherein the first part and second part taper to spaced-apart points at the second end.

12. The device of claim 11, where a distance between the spaced-apart points is about 3 mm to about 13 mm.

13. The device of claim 1, wherein the device is formed of a material selected from the group consisting of 304 stainless steel, 316 stainless steel, SAE 316 stainless steel, titanium or titanium alloys, Inconel, low carbon steel, alloys of steel including chromium, nickel, molybdenum, aluminum, austenitic stainless steel, and a combination of any two or more of the foregoing materials.

14. The device of claim 1, wherein the handle portion has a first length, wherein the screw engaging portion has a second length, wherein the first length is greater than the second length.

15. A device, comprising:
an elongated body having a first end comprising a handle portion and a second end comprising a screw engaging portion;
wherein the first end is disposed at an angle to the second end;
wherein the screw engaging portion comprises a bifurcated protrusion that comprises a first part and a second part, the first and second parts defining a generally V-shaped groove;
wherein the screw engaging portion defines a plane, wherein one of the first or the second part is coextensive of the plane, one of the first or the second part is not coextensive of the plane.

16. The device of claim 15, wherein the protrusion has an exterior surface that is biocompatible.

17. The device of claim 16, further comprising an impact-receiving element disposed on the distal end of said handle portion configured to receive forcible contact from an impact apparatus.

18. A method for extracting a solid screw from a bone; the method comprising extracting the solid screw from the bone using the device of claim 1.

19. The method of claim 18, wherein the method comprises:
engaging the solid screw in the V-shaped groove of the device of claim 1; and
applying a manual force to the first end of the elongated body to extract the solid screw from the bone.

20. The method of claim 19, further comprising, prior to the applying the manual force to the first end of the body, using at least one of the first part or the second part to dislodge tissue proximate the solid screw.
